(19) United States
(12) Patent Application Publication
(10) Pub. No.: US 2010/0089774 A1
(43) Pub. Date: Apr. 15, 2010

(54) NON-ENZYMATIC ELECTROCHEMICAL METHOD FOR SIMULTANEOUS DETERMINATION OF TOTAL HEMOGLOBIN AND GLYCATED HEMOGLOBIN

(75) Inventors: Venkat Manohar, Mumbai (IN); George Varghese, Mumbai (IN); Yegnaraman Venkatraman, Karaikudi (IN); Phani Lakshminarasimha Kanala, Karaikudi (IN); Mathiyarasu Jayaraman, Karaikudi (IN)

Correspondence Address: LADAS & PARRY LLP, 26 WEST 61ST STREET, NEW YORK, NY, 10023, US

(73) Assignee: PIRAMAL LIFE SCIENCES LIMITED

(21) Appl. No.: 12/366,747

(22) Filed: Feb. 6, 2009

(30) Foreign Application Priority Data
Oct. 14, 2008 (IN) 2200/MUM/2008

(51) Int. Cl. G01N 27/26; G01N 33/487

(52) U.S. Cl. 205/792; 204/403.01; 204/403.15; 204/403.02

(57) ABSTRACT

A non-enzymatic electrochemical method of simultaneous measurement of hemoglobin (Hb) and percentage of glycated hemoglobin (% HbA1c) in a blood sample is disclosed. The method includes determining the total amount of hemoglobin in a sample by electrochemically measuring the voltammetric current due to iron (II) and iron (III) redox portions in hemoglobin and determining the percentage of glycated hemoglobin (HbA1c) by potentiometry. Also disclosed is a novel screen-printed electrode (SPE) strip modified for potentiometric measurement of HbA1c.

### FIELD OF THE INVENTION

The present invention relates to a method which uses a non-enzymatic, disposable screen-printed electrode strip (SPE strip) for simultaneous measurement of total hemoglobin (Hb) and percentage of glycated hemoglobin (% HbA1c) in a blood sample wherein the total Hb is estimated by amperometry or differential pulse voltammetry, and the amount of HbA1c is estimated by potentiometry. Modification of a SPE strip for potentiometric measurement of HbA1c is also disclosed.

### BACKGROUND OF THE INVENTION

The importance of diagnosis and monitoring of diabetes is emphasized by a recent report in which it was stated that 20% of the total world population is affected by this chronic disease. One of the proactive measures needed to control diabetes mellitus is periodic monitoring and control of blood glucose levels either with the help of clinicians or using “do-it-yourself” kits. HbA1c is a stable minor variant of Hb, formed in vivo by non-enzymatic post-translational modification of N-terminal valine of the β-chains of Hb. Estimation of HbA1c is extremely valuable for long-term control of diabetes mellitus unlike direct estimation of glucose wherein one obtains information of blood sugar at the time of measurement. Hence, in addition to the monitoring of blood glucose levels, it is extremely important that one monitors the overall level of glucose by monitoring HbA1c. This is a better way to manage diabetes, and may result in the prevention or reduction of long-term complications. In recent years, various types of kits for monitoring HbA1c levels in blood have been described or developed.

U.S. Pat. No. 7,005,273 describes enzyme catalyzed electrochemical methods to measure Hb and HbA1c, and a spectrophotometric method to measure HbA1c. The method is based on an indirect electrochemical estimation of Hb using a measurement of dissolved oxygen and enzyme-catalyzed reactions. Disadvantages of this method relate to the stability of the enzyme and the shelf life of the system. It is well known that the dissolved oxygen levels are temperature dependent and hence a constant temperature environment needs to be maintained for the reliability of the analysis. Further, oxygen solubility in an aqueous environment is not sufficient to provide the required current signals for the indirect determination of Hb.

U.S. Pat. No. 6,677,158 describes a colorimetric method for HbA1c estimation that can be performed outside of the medical laboratory and includes several steps involving chemical addition and colour read-out devices for Hb measurement which require high dilution of the sample. This technique is rather complex and requires several manual operations. Moreover, in colorimetric measurements, sensitivity is relatively less compared to other methods.

U.S. Pat. No. 4,876,205 describes a method for assaying Hb in blood in which the blood is contacted with a sufficient amount of a ferricyanide (redox mediator) so that hemoglobin in the blood is reacted therewith and the hemoglobin is electrochemically assayed by monitoring the change in current, produced on reduction of ferricyanide by hemoglobin. The assay method incorporates a dry strip sensor with a dry mixture containing finely divided ferricyanide and a non-ionic surfactant, clerol (a mix of polyethylene oxide and polypropylene oxide and emulsifiers). However, this is a method useful only for total hemoglobin in whole blood. It is an indirect estimation of Hb and it has certain limitations, such as the dependence of the current signal on the kinetics of the redox transformations of the mediator. The use of redox mediators is not cost-effective for commercialization of the process.

EP 1,225,449 A1 describes the use of a non-enzymatic disposable electrode strip for detection of uric acid and Hb. The strip contains non-ionic or neutral surfactants such as Triton X-100 for Hb and a cationic surfactant for uric acid. The strip is used subsequently as an amperometric sensor. Neither anionic nor cationic surfactants are used in this method for sensing Hb.

There are known methods for analysis of HbA1c. For example, the DCA2000 analyzer from Siemens Diagnostics is an automated enzyme immunoassay method for determination of HbA1c. Most of the commercially available analyzers employ HPLC as a tool for the assay of HbA1c [Clinical Biochemistry, 2005, 38, 88-91]. There has been a report of use of a quartz crystal biosensor for detection of HbA1c using complexation reactions of diol groups with 3-aminophenylboronic acid [Analytica Chimica Acta, 2005, 530, 75-84].

There are reports about exploring electrochemical methods such as amperometry and variants to develop disposable sensors for determination of HbA1c. [Biosensors and Bioelectronics, 2006, 21, 1952-1959; Biosensors and Bioelectronics, 2007, 22, 2051-2056; Sensors and Actuators B, 2006, 113, 623-629; Sensors and Actuators A, 2006, 130-131, 267-272; Clinical Biochemistry, 2008, CLB6720, doi: 10.1016/j.clinbiochem.208.01.113].

The clinical estimation of HbA1c based on enzymatic conversion is rather complicated and requires the use of analytical methods such as cation exchange chromatography, affinity chromatography, gel electrophoresis, immunochemical and other spectroscopic methods. These techniques are complex, reagent-intensive and time-consuming. The cost per analysis is also relatively high. Though several methods for estimation of HbA1c are commercially available, there is a need for quick, robust and cost effective diagnostic tool for the analysis of HbA1c so that decisions can be made for better management of diabetes mellitus and complications thereof.

Therefore, an aspect of the present invention is to provide a rapid, non-enzymatic and direct method for simultaneous determination of HbA1c by potentiometry and total Hb by amperometry or differential pulse voltammetry in blood in a single analysis.

### SUMMARY OF THE INVENTION

The present invention relates to a screen-printed electrode (SPE) strip for simultaneous measurement of total Hb and % HbA1c in a blood sample. The strip includes four electrodes.

In one aspect of the invention, the SPE strip is non-enzymatic.

In another aspect of the invention, the SPE strip is disposable.

The invention also relates to a non-enzymatic, disposable screen-printed electrode (SPE) strip for simultaneous measurement of total Hb by amperometry or differential pulse voltammetry, and % HbA1c by potentiometry in a blood sample.

Still another aspect of the invention is that the strip is used in a method for simultaneous measurement of total Hb and % HbA1c in a blood sample.

The present invention also relates to a kit for simultaneous measurement of total Hb and % HbA1c in blood sample comprising a SPE strip (as described above), a lysis solution, and a surfactant solution. The kit may also include a lancet, a blotting paper strip, an empty vial and an instruction insert.

### BRIEF DESCRIPTION OF THE DRAWINGS

FIG. 1 is a block diagram of the SPE strip and its connection to a meter.

FIG. 2 is a block diagram of the hardware and the functional details of the meter.

FIG. 3 is a diagram of a screen-printed electrode (SPE) strip.

FIG. 4A shows a typical calibration plot for Hb by amperometry.

FIG. 4B shows the electrode response for Hb by amperometry.

FIG. 5A shows a typical calibration plot for Hb by differential pulse voltammetry (DPV).

FIG. 5B shows the electrode response for Hb by differential pulse voltammetry (DPV).

FIG. 6 shows the DPV response of Hb in 1.5 mM of Sodium dodecylsulphate (SDS) in acetate buffer of pH 5.0 [Hb conc. 0.7-1.7 g/dl].

FIG. 7 shows the potentiometric estimation of HbA1c (the graph line having square symbols ▪) using aminophenylboronic acid polymer film on the electrode surface and estimation of Hb (the graph line with triangle symbols ▴).

FIG. 8 shows the potentiometric estimation of HbA1c using aminophenylboronic acid in solution.

FIG. 9 shows the potentiometric estimation of HbA1c by using an electrode that has been modified with carbon ink using water-insoluble 4-phenyl-vinyl boronic acid (the graph line having square symbols ▪) and an electrode that has been modified with carbon ink using 3-thiophene boronic acid (the graph line with triangle symbols ▴).

### DETAILED DESCRIPTION OF THE INVENTION

### DEFINITIONS

Before describing the present invention in detail, it has to be understood that this invention is not limited to particular embodiments. It is also to be understood that the terminology used herein is for the purpose of describing particular embodiments only, and is not intended to be limiting.

As used in the specification and claims, the singular forms “a”, “an” and “the” include plural references unless the context clearly indicates otherwise.

The term “screen printed electrode (SPE) strip” refers to an electrode strip described below. It is to be understood that although the electrodes can be formed by using screen-printing, the invention is not limited to the use of screen-printing to form the electrodes. Other printing methods or other methods to form the electrodes can be used.

The term “Nemnstian response range” refers to a range of concentration in which the slope (defined by mV/decade of concentration) is less than the “ideal” Nernstian slope of 59 mV/decade.

The term “modified electrode” refers to an electrode whose surface is coated with layers of the desired functional materials specific to the application. In an embodiment of this invention, a screen-printed carbon or graphite electrode is modified by a water insoluble boronic acid compound.

The term “differential pulse voltammetry” refers to an electro-analytical technique in which a square wave pulse superimposed on a potential dc ramp (linear increase of potential with time) is applied on the sensing electrode and the differential current output is plotted against the applied dc potential.

The term “reaction area” refers to the area on the electrode, which is exposed to the blood sample.

The term “Glassy carbon” also called vitreous carbon refers to a non-graphitizing carbon, which combines glassy and ceramic properties with those of non-graphitizing carbon. The most important properties are high temperature resistance, extreme resistance to chemical attack and impermeability to gases and liquids. Glassy carbon is widely used as an electrode material in electrochemistry.

The term “meter” refers to an instrument, which measures potential difference and current signal generated at the electrode surface when the electrode comes in contact with the blood sample. The concentration of HbA1c is converted into potential difference, the concentration of Hb is converted into current signal, and both Hb and % HbA1c values are displayed on the screen of the meter.

Measurement of Total Hb

Hb consists of four protein chains with four heme portions (Fe²⁺/Fe³⁺) and is located in the erythrocytes. While not being bound by any theory, the approach of this invention involves analyzing Hb by exploiting the redox behaviour of the heme portions (Fe²⁺/Fe³⁺) in Hb molecule using a disposable, screen printed electrode surface coated with a material such as carbon, graphite, gold, platinum, palladium, or a printing ink as described below.

The method according to this invention includes determining the total amount of Hb in a sample by electrochemically measuring the voltammetric current due to iron (II) and iron (III) redox centers in Hb using surfactant-enhanced current signal amplification methodologies. The electrode potential is fixed at a level where the heme molecule interacts with the electrode surface to undergo electron transfer reaction. Thus the current observed is directly proportional to the amount of heme present, which in turn is related to the concentration of total Hb present in the given test solution. As the heme centers in Hb are buried deep into the bulky protein molecules, it is difficult to get an appreciable current signal. To overcome this, heme portions should be released or made available before performing amperometric measurement. For this purpose, the Hb is treated as described below with a current-enhancing surfactant.

The released heme group shows significant redox characteristics at the electrode without a redox mediator. The heme group can also be released from the Hb molecule using sonication followed by centrifugation or by providing the Hb molecule a chemical link to redox mediators (such as ferrocene, methylviologen, etc.). The current signal can thus be amplified by using the ionic surfactant and is converted to g/dL of Hb and displayed on the screen of the meter.

Measurement of HbA1c

HbA1c is the glycated form of Hb resulting from the condensation reaction between hexose sugars and Hb. In the present invention, HbA1c has been analyzed using a potentiometric approach unlike optical, redox-mediated amperometry, immunoassay and other methods such as quartz crystal mass balance methods.

The water-insoluble boronic acid compound added as described below results in a complex being formed between boronate and the cis-diol groups of sugars present in the HbA1c. During these chemical changes, an equilibrium potential of the electrode surface is developed that depends on the HbA1c concentration in the sample. The potential difference arises due to change in pKa value of the boronic acid compound at the electrode surface. This results in a linear relationship between HbA1c concentration and the potential difference measured by making use of the sub-Nernstian response range of the potentiometric technique. This potential difference is measured with respect to the reference electrode and is converted into % HbA1c and displayed on the screen of a meter.

After the SPE strip (which is connected directly or indirectly to a meter) is wet by the solution containing red blood corpuscles (RBCs), the presence of Hb and HbA1c is detected by amperometry or differential pulse voltammetry, and potentiometry respectively. The current signal can be amplified by using an ionic surfactant, which is converted to g/dL of Hb and displayed on the screen of the meter.

Description of the SPE Strip

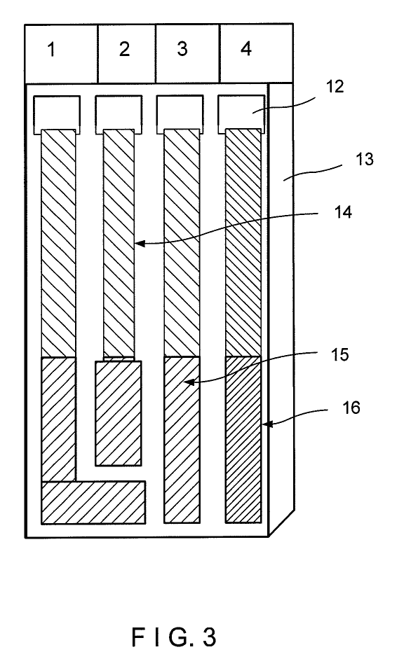

The SPE strip includes contact pads that are the upper portion of the electrodes and are illustrated by 12 in FIG. 3; insulating material, which is the substrate, and insulating non-porous film, which is the electrical insulating film. The strip, which may be disposable, is used for non-enzymatic detection of Hb and % HbA1c. It comprises:
(i) A substrate, which is an electrical insulator. Types of electrical insulators that can be used include but are not limited to glass epoxy board; electrically non-conducting polymer material such as polystyrene; or fiber-reinforced epoxy (FRE) substrates of thickness varying from 0.3 mm to 1.0 mm. In an aspect of the invention, the substrate is FRE.
(ii) A conducting film, which is coated on one side of the substrate to form four independent electrodes, namely, (a) counter electrode, (b) working electrode, (c) reference electrode and (d) modified electrode.
(iii) An electrical insulating film. The electrical insulating film is coated on a part of the conducting film such that one end of all the electrodes are uncovered for connecting with the measuring device and the opposite end is uncovered and is intended to be in contact with the solution containing the sample to be tested. The electrical insulating film has properties of electrical insulation with very high impedance of greater than 10¹² ohms. This material is used to coat the conducting film to provide the electrical insulation. The electrical insulating film can be a commercially available material. An example of an electrical insulating film is XV1300U.V.—NOTATION WHITE“, INK NO. CFSN6022, supplied by Sun Chemical, UK.

In an aspect of the present invention, the SPE strip comprises four electrodes wherein electrode 1 (counter electrode), electrode 2 (working electrode) and electrode 3 (reference electrode) are used for estimation of Hb by amperometry or differential pulse voltammetry; and electrode 3 and electrode 4 (modified electrode) are used for estimation of HbA1c by potentiometry. Electrode 3 is a common reference electrode for both amperometry and potentiometry. The electrodes are independent of each other and do not touch each other. Electrodes 1, 2, 3 and 4 are shown in FIG. 3.

According to an aspect of the invention, the locations for the electrodes are marked and one side of the substrate is coated with a conducting film using screen-printing or a similar printing method to form the electrodes. Other methods can also be used to form the electrodes. In this process only the electrodes are coated and not the entire substrate.

The conducting film is selected from gold, platinum, palladium, silver, carbon or graphite or a printing ink which has the property of adhering to the surface of the substrate without any smearing so that the electrodes remain independent of each other. The conducting film accepts or donates electrons and can be used as the mediator to transfer electrons between the analyte and the electrode in the redox reaction.

In an embodiment of the invention, printing ink is used as the conducting film and the printing ink typically used is a carbon or graphite ink or a mixture of a carbon and silver ink. In an aspect of the present invention, the material for coating the electrodes is a carbon conducting film or carbon printable ink. Any commercially available conductive carbon ink which gives an electrochemical response for standard cyclic voltammetry experiments can be used. A material that can be used for coating the substrate using screen-printing is a conductive carbon paste procured from Coates, Inc. (USA). This conductive carbon paste can be used as an ink to print on predetermined areas of the substrate to form the electrodes.

In one aspect of the invention the thickness of the conducting film on the substrate is between 20 to 60 microns. In another aspect of the invention, the thickness of the conducting film is about 30 microns.

The range of the dimensions of each electrode of the SPE strip, which is exposed to the solution containing the red blood corpuscles (RBCs) (region 15 in FIG. 3), may be:

Electrode 1 (Counter Electrode):

Length—3.0 mm to 10.0 mm, preferably 5.0 mm

Width—0.3 mm to 2.0 mm, preferably 0.5 mm

Thickness—20 microns to 150 microns, preferably 60 microns.

Electrode 2 (Working Electrode):

Length—2.0 mm to 9.0 mm, preferably 4.0 mm

Width—0.3 mm to 2.0 mm, preferably 1.0 mm

Thickness—20 microns to 150 microns, preferably 60 microns.

Electrode 3 (Reference Electrode):

Length—3.0 mm to 10.0 mm, preferably 5.0 mm

Width—0.3 mm to 2.0 mm, preferably 0.5 mm

Thickness—20 microns to 150 microns, preferably 60 microns.

Electrode 4 (Modified Electrode):

Length—3.0 mm to 10.0 mm, preferably 5.0 mm

Width—0.3 mm to 2.0 mm, preferably 0.5 mm

Thickness—20 microns to 150 microns, preferably 60 microns.

After the substrate is coated with the conducting film, it is dried at a temperature from 90° C. to 150° C., preferably at about 120° C., for about 30 minutes to 60 minutes, preferably for about 45 minutes. After drying, the substrate is dipped in an acid. Examples of acids that can be used are 10% chromic acid, 10% sulfuric acid, 5-10% nitric acid or 10% hydrochloric acid solution for 10.0 minutes. In an aspect of the invention, the coated substrate is dipped in 10% chromic acid solution. The substrate is removed from the chromic acid solution and washed with water three times for 2 to 15 minutes per wash, preferably, about 10 minutes per wash. The substrate is again dried, preferably at about 70° C. for about 20 minutes.

An electrical insulating film is applied to the strip by screen printing or another method except on the contact pads and the section of the strip identified as region 15 in FIG. 3.

The conducting film of the fourth electrode (modified electrode), is modified by a water-insoluble boronic acid compound using screen printing or the like at the portion of the electrode that will be immersed in the sample of RBCs shown as 16 in FIG. 3. This modified coating enables changes such as potential, resistance by electrochemical reaction between the modified electrode and reference electrode to be used to determine the % HbA1c.

Electrode modification is not possible with the soluble form of boronic acid compounds because the electrode will lose its sensing ability due to the leaching of HbA1c-selective boronic acid and the associated functional groups. Thus, in the present invention, water-insoluble boronic acid compounds have been used to modify the fourth electrode (electrode 4). The water-insoluble boronic acid compound may be selected from 4-phenyl-vinyl boronic acid, aminophenyl boronic acid and thiophene boronic acid. In one aspect of the invention 4-phenyl vinyl boronic acid is used.

The fourth electrode can be modified according to the following procedures:

(a) A water-insoluble boronic acid compound is dissolved in a suitable low volatile solvent that can dissolve the water insoluble boronic acid compound. The solvent may be selected from isopropyl alcohol, ethanol, propanol and acetone. The solution obtained can be blended with the conductive carbon paste in a weight ratio of 1:0.5 to 1:4, preferably in a ratio of about 1:1 and used for printing on the substrate for potentiometric estimation of HbA1c.

(b) In an alternative configuration, (for potentiometry) the printed carbon electrode is modified with a film (thickness: approx.5-10 μm) of a water-insoluble boronic acid compound, by electro-deposition on the carbon electrode using electro-polymerization procedure/conditions. The water-insoluble boronic acid compound and sodium fluoride are dissolved in hydrochloric acid solution. Polymerization is effected by dipping the screen-printed fourth carbon electrode in this solution without stirring. The fourth electrode potential is scanned between 0.0 and 1.1 V until the charge in the cathodic scan reaches 10 mC cm⁻². A deep bluish-green film is obtained and it is washed with water. The electrode is thus modified and then rinsed with water, followed by rinsing in phosphate buffered saline (PBS) solution.

Other processes can be used to prepare the modified electrode.

Only the portion of the fourth electrode that will be immersed in the sample of RBCs is modified.

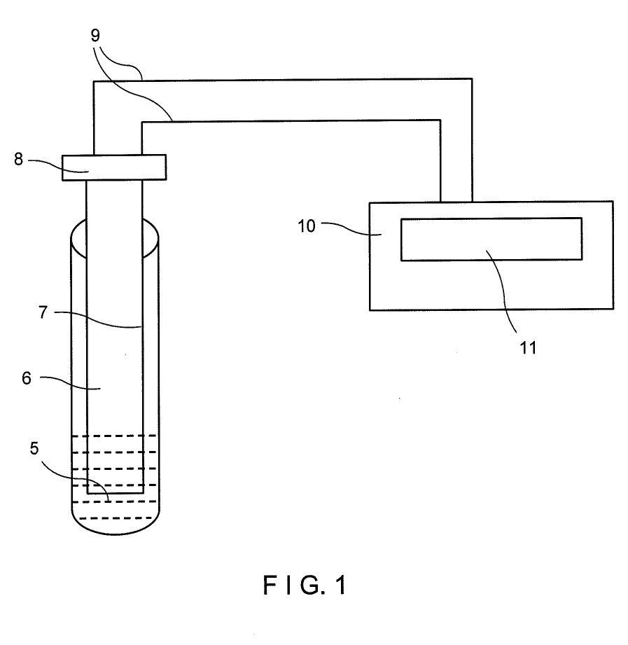

FIG. 3 describes a screen-printed electrode (SPE) strip. It consists of four electrodes, namely, counter electrode 1, working electrode 2, reference electrode 3 and modified electrode 4. Basically, the electrodes are screen printed on the substrate 13 using a conducting film. Preferably, the conductive carbon ink of resistance in the range 15 ohms to 25 ohms is used to screen print the electrodes 1, 2, 3 and 4 on substrate 13. Contact pads 12 are at the top end of the electrodes and are used to provide the electrical connection with the connector 8 in FIG. 1. Preferably, the width of the contact pads is the same for all four electrodes. An electrically insulating film 14 is screen printed on all the electrode surfaces except for the contact pads and the section of the electrodes identified as region 15. Region 15 is the portion of the electrodes that come in contact with the sample containing the RBCs (5 in FIG. 1) for determination of concentration of hemoglobin and glycated hemoglobin. Only the portion of electrode 4 that is to be immersed in the sample is modified using a water insoluble boronic acid compound and is shown as 16 in FIG. 3.

Additionally, the invention also relates to a non-enzymatic, electrochemical method for simultaneous measurement of total Hb and % HbA1c in blood sample using the SPE strip (as described above) comprising the steps of:

(a) treating a blood sample with a lysis solution;

(b) removing the plasma from the blood sample to obtain red blood corpuscles (RBCs);

(c) treating the sample containing the RBCs obtained in step (b) with a surfactant solution;

(d) contacting the sample obtained in step (c) with the SPE strip;

(e) measuring of total Hb by amperometry or differential pulse voltammetry; and measurement of HbA1c by potentiometry; and

(f) calculating the % HbA1c relative to the total Hb in blood sample.

The blood sample collected from the patient is subjected to pre-treatment to separate red blood corpuscles (RBCs) from plasma by adding a lysis solution. Plasma can be removed from the blood sample using different techniques or methods. Non-limiting ways that plasma can be removed include decanting or by dipping a blotting paper in the blood sample with lysis solution and the RBCs obtained are treated with the surfactant solution.

The lysis solution may be selected from 50% ethanol; 1M acetic acid (in water) 0.2M acetic acid (in water) 0.2M citric acid (in water); ethyl alcohol/water (1:1) and NaCl (in water).

The ratio of the lysis solution to the sample is 1:1 to 1:20 (v/v), preferably, 1:10 (v/v).

The surfactant may be selected from all types of cationic, anionic, e.g. ionic surfactants and preferably is selected from gemini surfactants, didodecyldimethylammonium bromide, cetyltrimethylammonium bromide, benzyltrimethylammonium bromide, phenacylthiazolium bromide, aminoguanidine hydrochloride, thiourea, phenacyl-thiazolium/-pyridinium bromide, sodium dodecylsulfate, sodium polystyrenesulfonate, and sodium salts of benzene-/naphthalene-mono-/di-/tri-sulfonic acids.

The ratio of the surfactant to the sample of RBCs is 1:1 to 1:20 (v/v) and preferably 1:10 (v/v).

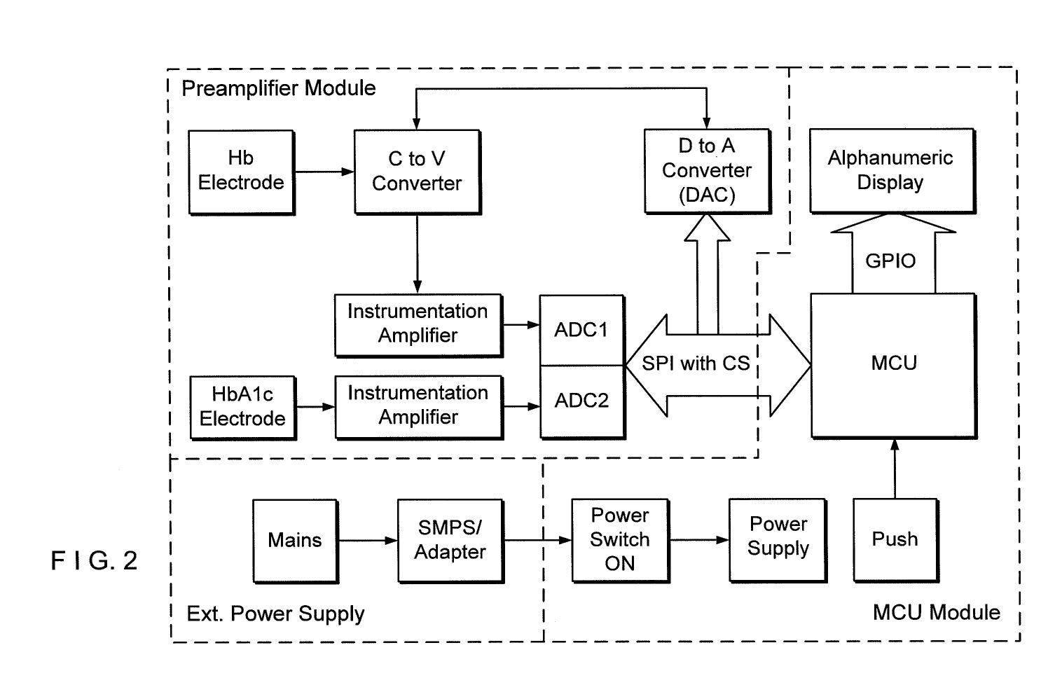

The SPE strip is introduced into the sample containing treated RBCs. A potential difference is generated due to reaction of HbA1c on the surface of the boronic acid modified electrode. This potential difference is measured with respect to the reference electrode and is converted into % HbA1c and displayed on the screen of a meter. Similarly, a current signal is generated between electrodes 1, 2 and 3 proportional to the concentration of hemoglobin wherein the Fe²⁺/Fe³⁺ reaction takes place on the electrode surface. The current signal is converted to g/dL of Hb and displayed on the screen of the meter. The functional details of the meter are shown in FIG. 2. The dotted line separates the components of the Printed Circuit Board (PCB) comprising a preamplifier and Microcontroller Unit (MCU) modules. The Hb electrodes (electrode 1, 2 and 3) generate the current signal, which is subsequently converted into equivalent voltage signal through a current to voltage converter. The modified electrode directly generates a potential difference, which in turn is measured as a voltage signal. Both the voltage signals corresponding to Hb and HbA1c respectively are amplified through Instrumentation Amplifier. The Analog to Digital Converter (ADC) converts the amplified analog voltage signals to equivalent digital signals. The MCU processes the digital data and directly displays the Hb value in terms of g/dL and HbA1c as a percentage value on Alphanumeric Display.

In an aspect of the present invention, both the values of total Hb and HbA1c are required to calculate the value of % HbA1c. The percentage of HbA1c is calculated as follows:

% HbA1c=[(HbA1c/total Hb)×100].

The entire analysis may be completed within five to ten minutes after collection of the blood. As shown, for example, in FIG. 7, according to the invention, Hb and HbA1c can each be measured and quantified and there is no interference between the measurements and quantification of each as it pertains to the other.

FIG. 1 shows block diagram of how a typical analysis is carried out by connecting the SPE strip with the meter. The sample in vial 6 contains red blood corpuscles (RBCs) 5, which have been isolated from plasma. The surfactant solution, preferably an ionic surfactant solution is added to vial 6 to preferentially release Heme proteins. The RBCs are mixed with the surfactant solution and can be analyzed. The SPE strip 7 is connected to the connector end 8 of the meter 10, through the cable 9. The sensor measures the concentration of Hb and HbA1c in the vial, the MCU calculates both Hb and HbA1c in g/dL and % unit respectively. The meter 10 indicates these values on the display 11.

The present invention also relates to a kit for simultaneous measurement of total Hb and % HbA1c in blood sample comprising a SPE strip (as described above), a lysis solution, and a surfactant solution. The kit may also include a lancet, a blotting paper strip, an empty vial and an instruction insert.

In one embodiment of the present invention, the lancet is used for pricking the skin so the blood can be collected in the empty vial.

The instruction insert provides instructions for use of the kit. The insert may include instructions describing the steps needed to measure Hb and % HbA1c in the sample including describing how the blood is drawn, and mixed with the lysis and surfactant solutions.

The invention thus provides a method for the estimation of % HbA1c and total Hb in a single step using a disposable, non-enzymatic screen-printed electrode strip, which incorporates electrodes for amperometry or differential pulse voltammetry and potentiometry.

An example of an apparatus that can be used is a tabletop device that can be used in a medical practitioner's office. In some embodiments, an apparatus that can be used may be operated by non-technically trained people.

The above disclosure generally describes the present invention. More details of the above invention can be understood from the following specific examples. These examples are herein provided for the purpose of illustration only and are not intended to limit the scope of the invention.

### EXAMPLES

### Example 1

Preparation of Electrodes

Starting material used for the preparation of electrodes of the screen-printed sensor strip was conductive carbon paste procured from Coates, Inc. (USA). This conductive carbon paste was used as an ink to print on the predetermined areas of the fibre-reinforced epoxy (FRE) substrates using a screen-printing process.

### Example 2

Modification of Electrode 4

As shown in FIG. 3, region 16 of electrode 4 of the SPE strip prepared in Example 1 was modified by dissolving 4-vinylphenyl boronic acid in iso-propyl alcohol (˜10 ml) and blended with the conductive carbon paste in 1:1 ratio (by weight) and was used for screen printing for potentiometric estimation of HbA1c from blood sample.

### Example 3

Process for Modification of Electrode 4

In this process, the screen-printed carbon electrode of Example 1 was modified with a conducting polymer film (thickness: approx.5-10 μm) of amino phenyl boronic acid (PABA). It was electro-deposited on the carbon electrode using the electro-polymerization procedure/conditions, which are briefly described as follows: 3-amino phenyl boronic acid (0.04 M) of quantity 87.0 mg and sodium fluoride (0.2 M) of quantity 105.0 mg were dissolved in 12.5 ml of 0.2 M HCl solution. Polymerization was effected by dipping one of the screen-printed carbon electrodes in the above solution under unstirred conditions and the electrode potential was scanned between 0.0 and 1.1 V until the charge in the cathodic scan reached 10 mC cm⁻². A deep bluish-green film was obtained and it was washed with water. The electrode was thus modified and then rinsed with water, followed by PBS solution and it was ready for use.

### Example 4

Calibration Curve for Estimation of Hb by Amperometry Using Didodecyldimethyl Ammonium Bromide (DDDMAB) as a Surfactant.

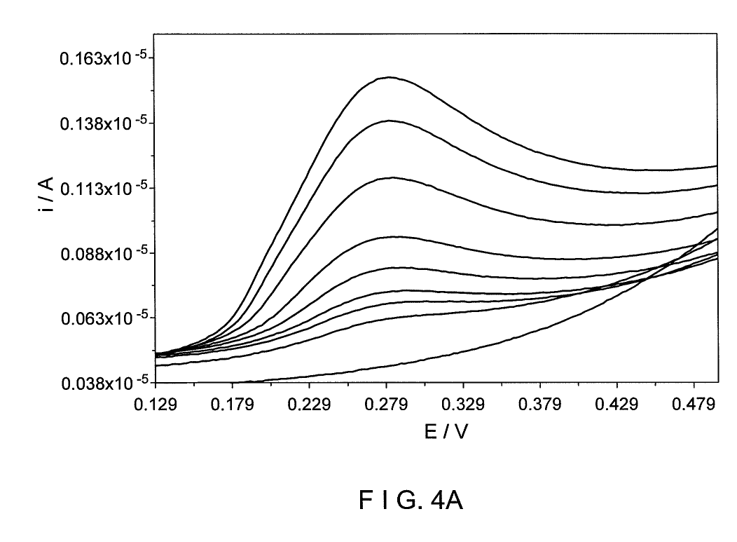

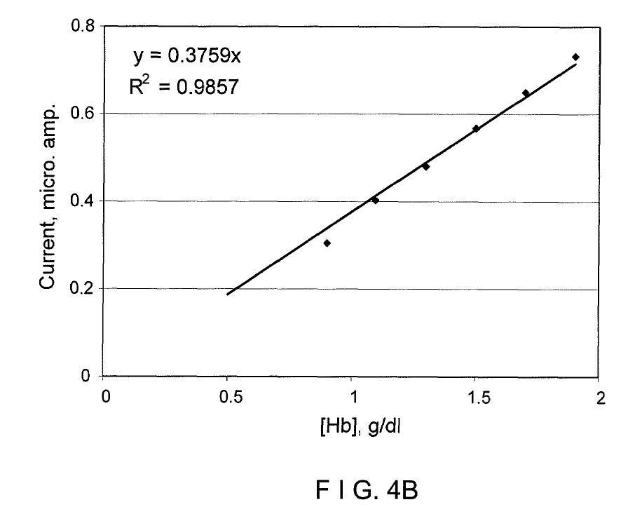

The standard hemoglobin sample (Catalog No. 400294022, Nicholas Piramal India Limited) (15 g/dl) was diluted ranging from concentration of 0.5 g/dl to 1.9 g/dl using the surfactant solution containing DDDMAB dissolved in 0.1M potassium chloride solution. The SPE strip, prepared in Example 1, was introduced into the above sample solution. Then the electrodes were connected to the potentiostat using appropriate connectors and the potential was swept between 0.1 to 0.8 volt at a scan rate of 100 mV/s. The peak current was measured in the peak potential range of 0.25 to 0.30 V. This was repeated with five standard samples and a calibration plot of “peak current vs. Hb concentration” was plotted. From the calibration plot, the slope of the graph was calculated and the latter was used for determination of total Hb in the test sample. A typical calibration plot and the electrode response for Hb in 5 mM DDDMAB-1M KCl solution is shown in FIGS. 4A and 4B.

The experimental calibration graph for Hb, carried by amperometry, is linearly fitted by a straight line. The equation y=0.3759x gives the best fit with regression coefficient R²=0.9857. This equation is used to determine the concentration of Hb present in the sample.

### Example 5

Calibration Curve for Estimation of Hb by Differential Pulse Voltammetry Using DDDMAB as Surfactant

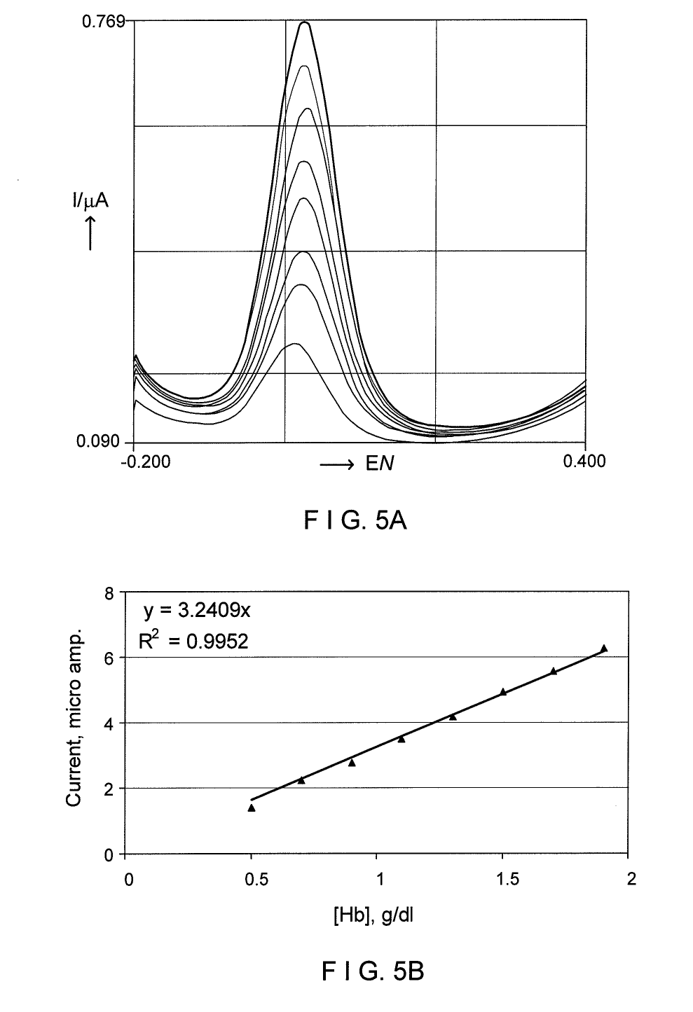

The standard hemoglobin sample (catalog no. 400294022, Nicholas Piramal India Limited) (15 g/dl) was diluted ranging from concentration of 0.5 g/dl to 1.9 g/dl using the surfactant solution containing didodecyldimethyl ammonium bromide (DDDMAB) dissolved in 0.1M potassium chloride solution. The SPE strip, prepared in Example 1 was introduced into the above sample solution. Then the electrodes were connected to the potentiostat using appropriate connectors in the differential pulse voltammetry (DPV) mode. The potential was swept between −0.2 and 0.4 V at a scan rate of 5 mV/s using the parameters: step potential: 2 mV; pulse width: 50 mV; pulse period: 200 ms. The DPV peak current was measured in the above potential range. This was repeated with five standard samples and a calibration plot of “peak current vs. Hb concentration” was plotted. From the calibration plot, the slope of the graph was calculated and the latter was used for determination of total Hb in the test sample. A typical calibration plot and the electrode response for Hb in 5 mM DDDMAB-1M KCl solution is shown in FIG. 5A and 5B.

The experimental calibration graph for Hb, carried by differential pulse voltammetry, is linearly fitted by a straight line. The equation y=3.2409x gives the best fit with regression coefficient R²=0.9952. This equation is used to determine the concentration of Hb present in the sample.

### Example 6

Calibration Curve for Estimation of Hb by Differential Pulse Voltammetry Using Sodium Dodecyl Sulphate as the Surfactant.

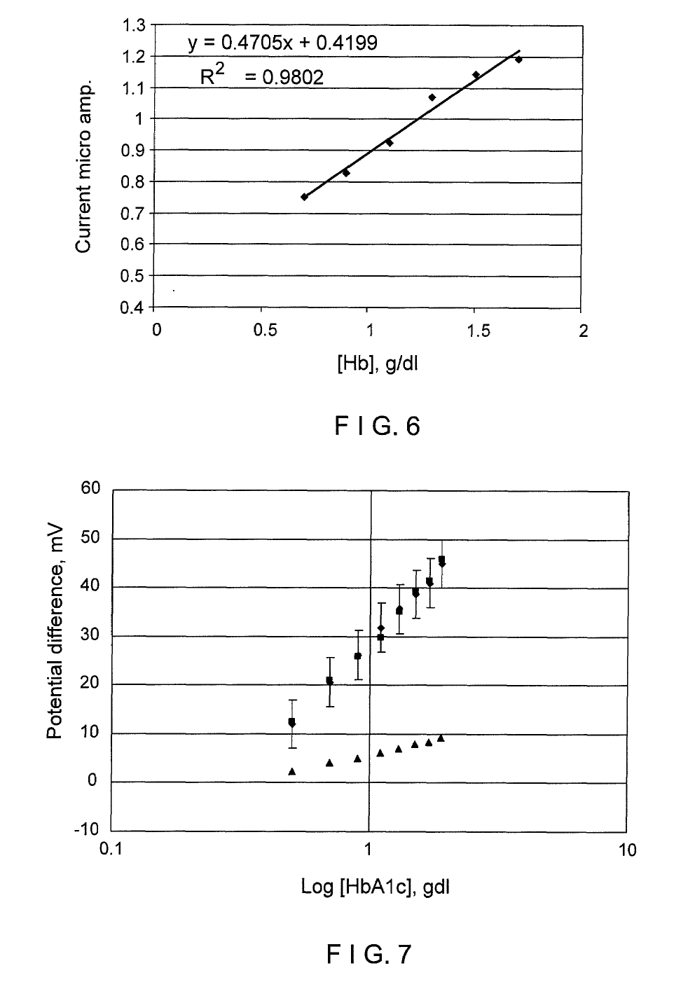

The standard hemoglobin sample (Catalog No. 400294022, Nicholas Piramal India Limited) (15 g/dl) was diluted ranging from concentration of 0.5 g/dl to 1.9 g/dl using the surfactant solution containing sodium dodecyl sulphate (SDS) dissolved in 0.1M potassium chloride solution. The SPE strip, prepared in Example 1, was introduced into the above sample solution. Then the electrodes were connected to the potentiostat using appropriate connectors and the potential was swept between 0.1 to 0.8 volt at a scan rate of 100 mV/s. The peak current was measured in the peak potential range of 0.25 to 0.30 V. This was repeated with five standard samples and a calibration plot of “peak current vs. Hb concentration” was plotted. From the calibration plot, the slope of the graph was calculated and the latter was used for determination of total Hb in the test sample. A 10 typical calibration plot and the electrode response for Hb in 5 mM SDS-1M KCl solution is shown in the FIG. 6.

The experimental calibration graph for Hb, carried by differential pulse voltammetry, is linearly fitted by a straight line. The equation y=0.475x+0.4199 gives the best fit with regression coefficient R²=0.9802.

### Example 7

Calibration Curve for Estimation of % HbA1c by Potentiometry Using SPE Strip Modified by Aminophenylboronic Acid.

A film of aminophenylboronic acid (PABA) was deposited on the glassy carbon electrode. 3-amino phenyl boronic acid (0.04 M) and sodium fluoride (0.2 M) were dissolved in hydrochloric acid (0.2M) solution. Polymerization was effected by keeping the working electrode in this solution along with platinum foil as counter electrode and saturated calomel as reference electrode. The electrode potential was scanned between 0.0 and 1.1 V for 3-5 scans. The modified electrode was then rinsed with water followed with PBS solution and used for further experiments.

Based on these results, a linear relationship was established between concentration of HbA1c and the potential difference, enabling potentiometric estimation of the HbA1c as shown in the FIG. 7.

The graph line in FIG. 7 having square symbols (▪) indicates the change in the potential difference of HbA1c as a function of concentration of HbA1c. The graph line in the figure with triangle symbols (▴) indicate the change in the concentration of Hb alone. The separation of these two graph lines show that that there is no interference from Hb in the detection and quantification of HbA1c when both Hb and HbA1c are measured simultaneously.

### Example 8

Calibration Curve for Estimation of % HbA1c by Potentiometry Using SPE Strip Modified by Water-Soluble Aminophenylboronic Acid.

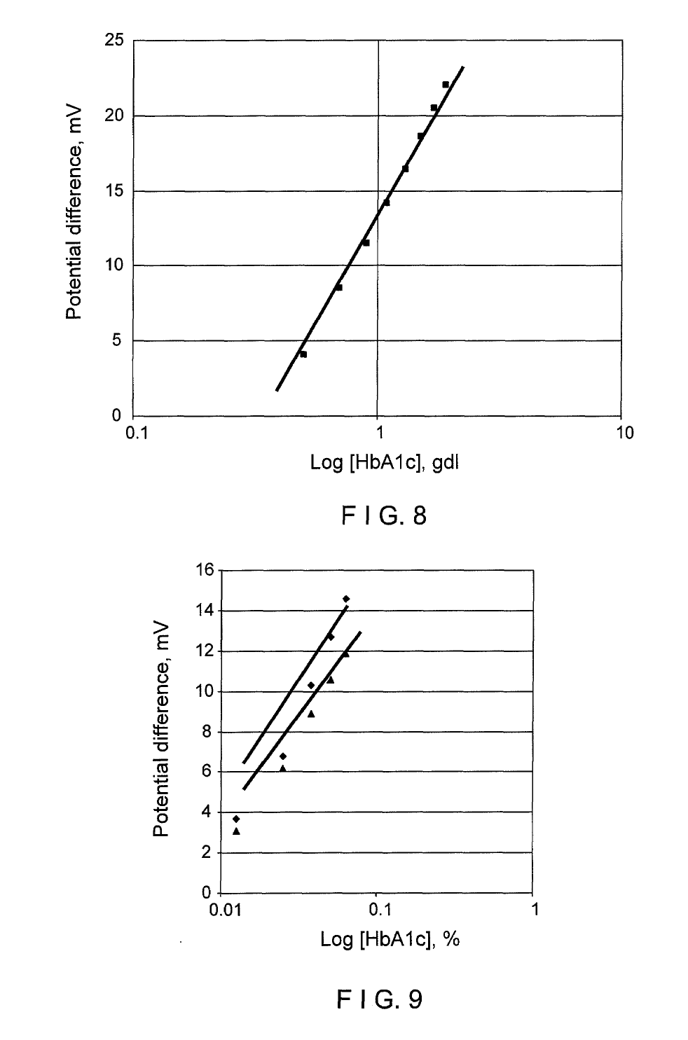

SPE strip, prepared in Example 1, was used for the experiment. Water-soluble lo aminophenylboronic acid (APBA) was dissolved in an electrolyte solution containing the sample and the consequent shift in electrode potential due to addition of HbA1c was measured. Aminophenylboronic acid in solution interacts with HbA1c, yielding a relationship between the concentration of HbA1c and the measured potential difference. This potential difference arises due to change in pKa value at the electrode surface. Based on these results, a linear relationship (FIG. 8) was established between concentration of HbA1c and the potential difference, enabling potentiometric estimation of HbA1c.

### Example 9

Calibration Curve for Estimation of % HbA1c by Potentiometry Using SPE Strip Modified by Vinylphenylboronic Acid.

This method estimates the potential of an electrode modified by a carbon ink of (water-insoluble) vinylphenylboronic acid, which was immersed in an electrolyte solution containing the sample (TruLab HbA1c liquid level 1 to level 4; Diagnostic System GmbH, Germany) and the consequent shift in electrode potential due to addition of HbA1c. This modified electrode interacts with HbA1c, yielding a relationship between the concentration of HbA1c and the measured potential difference. This potential difference arises due to change in pKa value at the electrode surface. Based on these results, a linear relationship was established between concentration of HbA1c and the potential difference, enabling potentiometric estimation of HbA1c as shown in the FIG. 9. This experiment demonstrates the linear relationship between HbA1c and potential difference.

FIG. 9 shows the potentiometric estimation of HbA1c by using an electrode that has been modified with carbon ink using water-insoluble 4-phenyl-vinyl boronic acid (the graph line having square symbols ▪) and an electrode that has been modified with carbon ink using 3-thiophene boronic acid (the graph line with triangle symbols ▴).

### Example 10

Calibration Curve for Estimation of % HbA1c by Potentiometry Using SPE Strip Modified by Thiopheneboronic Acid.

An electrode modified by a carbon ink of (water-insoluble) thiopheneboronic acid was immersed in an electrolyte solution containing the sample (TruLab HbA1c liquid level 1 to level 4 Diagnostic System GmbH, Germany) and the consequent shift in electrode potential due to addition of standard HbA1c was measured. This modified electrode interacts with HbA1c, yielding a relationship between the concentration of HbA1c and the measured potential difference. This potential difference arises due to changes in pKa value at the electrode surface. Based on these results, a linear relationship was established between concentration of HbA1c and the potential difference, enabling potentiometric estimation of HbA1c as shown in FIG. 9.

### Example 11

Measurement of Total Hb and % HbA1c from a Patient's Blood Sample.

A blood sample from a diabetic patient was collected at a clinical laboratory. 20 μL of blood sample was taken in a test vial and 200 μL of lysis solution consisting of 50% ethanol was added. The vial was kept for two minutes without shaking so that plasma was separated from RBCs. The separated plasma was decanted by tilting the vial. RBC, being a thick fluid did not flow out of the vial while decanting the plasma. Then, 200 μL of surfactant 5 mM ionic surfactant, cetyl trimethyl ammonium bromide (CTAB) was added and the solution was manually shaken approximately for a minute for mixing of RBC with the surfactant solution. The solution was ready for analysis.

The SPE strip modified by 4-phenyl-vinyl-boronic acid was inserted in the vial. A potential difference was generated due to reaction of HbA1c on the surface of boronic acid modified electrode which was measured with respect to the reference electrode and was converted into % HbA1c and displayed on the screen of the meter. Similarly, a current signal generated between electrodes 1, 2 and 3 proportional to the concentration of hemoglobin was converted to g/dL of Hb and displayed on the screen of the meter. The Hb concentration was 10.52 g/dl and the % HbA1c value was 9.3%. Sample from the same patient was analysed using Chloestech GDX A1C testing system and HbA1c was estimated to be 9.2%.

What is claimed is:

1. A screen printed electrode (SPE) strip for simultaneous measurement of total hemoglobin and percentage of glycated hemoglobin in a blood sample comprising four electrodes comprising a counter electrode, working electrode, reference electrode and an electrode modified by a water—insoluble boronic acid compound:
wherein the counter, working and reference electrodes are used for estimation of Hb by amperometry or differential pulse voltammetry; the reference and modified electrodes are used for estimation of HbA1c by potentiometry; and
the reference electrode is used for both amperometry and potentiometry.

2. The screen-printed electrode strip according to claim 1, wherein the electrodes are coated with a material selected from the group consisting of carbon, graphite, gold, platinum, palladium and silver.

3. The screen-printed electrode strip according to claim 2, wherein the electrodes are coated with carbon or graphite.

4. The screen-printed electrode strip according to claim 2, wherein the electrodes are coated with carbon.

5. The screen-printed electrode strip according to claim 4, wherein carbon is in the form of printable ink.

6. The screen-printed electrode strip according to claim 1, wherein the electrodes are formed by using printing.

7. The screen-printed electrode strip according to claim 1, wherein the strip is non-enzymatic.

8. The screen-printed electrode strip according to claim 1, wherein the strip is disposable.

9. The screen-printed electrode strip according to claim 1, wherein the boronic acid compound is selected from a group consisting of 4-phenyl-vinyl boronic acid, aminophenyl boronic acid and thiophene boronic acid.

10. The screen-printed electrode strip according to claim 1, wherein the strip is prepared by a process comprising the steps of:
a) coating with a conducting film on one side of the substrate to form the electrodes comprising of a counter electrode, working electrode, reference electrode and a modified electrode wherein the electrodes are isolated and disconnected;
b) washing the substrate in an acid solution and drying;
c) coating an insulating film on a part of the electrodes wherein one end of the electrodes is uncovered to make contact with the meter and the other end of the electrodes, which is opposite to the end which can be connected to the meter, is also uncovered;
d) modifying the portion of the electrode 4, which is opposite to the end which can be connected to the meter, using a water-insoluble boronic acid compound.

11. A kit for simultaneous measurement of total Hb and % HbA1c in blood sample comprising a SPE strip of claim 1.

12. The kit according to claim 11, further comprising a lysis solution and a surfactant solution.

13. The kit according to claim 12, further comprising a lancet, a blotting paper strip, an empty vial and an instruction insert.

14. The kit according to claim 11, wherein the lysis solution is selected from 50% ethanol; 1M acetic acid (in water) 0.2M acetic acid (in water) 0.2M citric acid (in water); ethyl alcohol/water (1:1) and NaCl (in water).

15. The kit according to claim 12, wherein the lysis solution is a premeasured amount of 50% ethanol.

16. The kit according to claim 12, wherein the surfactant solution is a premeasured amount of ionic surfactant selected from the group consisting of gemini surfactants, didodecyldimethylammonium bromide, cetyltrimethylammonium bromide, benzyltrimethylammonium bromide, phenacylthiazolium bromide, aminoguanidine hydrochloride, thiourea, phenacyl-thiazolium/-pyridinium bromide, sodium dodecylsulfate, sodium polystyrenesulfonate, and sodium salts of benzene-/naphthalene-mono-/di-/tri-sulfonic acids.

17. A method for simultaneous measurement of total Hb and % HbA1c in blood sample comprising:
(a) treating a blood sample with a lysis solution;
(b) removing the plasma from the blood sample to obtain red blood corpuscles (RBCs);
(c) treating the solution obtained in step (b) with a surfactant solution;
(d) contacting the solution obtained in step (c) with the screen printed electrode strip of claim 1;
(e) measuring total Hb by amperometry or differential pulse voltammetry; and measuring of HbA1c by potentiometry; and
(f) calculating the % HbA1c relative to the total Hb in the solution of red blood corpuscles.

18. The method according to claim 17, wherein the plasma is removed by decanting or by dipping a blotting paper strip in the sample.

19. The method according to claim 17, wherein the lysis solution is selected from 50% ethanol; 1M acetic acid (in water) 0.2M acetic acid (in water) 0.2M citric acid (in water); ethyl alcohol/water (1:1) and NaCl (in water).

20. The method of claim 17, wherein the surfactant solution is an ionic surfactant selected from the group consisting of Gemini surfactants, phenacyl-thiazolium/-pyridinium bromide, aminoguanidine hydrochloride, thiourea, dodecyldimethylammonium bromide, cetyltrimethylammonium bromide, benzyltrimethylammonium bromide, phenacylthiazolium bromide, sodium dodecylsulfate and sodium polystyrenesulfonate.
